(19) United States
(12) Patent Application Publication
(10) Pub. No.: US 2025/0352214 A1
(43) Pub. Date: Nov. 20, 2025

(54) Vascular Occlusion Device

(71) Applicant: Terumo Corporation, Tokyo (JP)

(72) Inventors: George Stern, Aliso Viejo, CA (US); Wendy Graczyk Beuthin, San Clemente, CA (US); Tin Luong-Uhl, Huntington Beach, CA (US); Emir Guevara, Aliso Viejo, CA (US); Rajesh Radhakrishnan, Aliso Viejo, CA (US)

(73) Assignee: Terumo Corporation, Tokyo (JP)

(21) Appl. No.: 18/871,418

(22) PCT Filed: May 30, 2023
(86) PCT No.: PCT/US2023/067634

§ 371 Date: Dec. 3, 2024

Related U.S. Application Data
(60) Provisional application No. 63/366,051, filed on Jun. 8, 2022.

(51) Int. Cl. A61B 17/12

(52) U.S. Cl. CPC A61B 17/12113; A61B 17/12172

(57) ABSTRACT

A vascular occlusion device for occluding blood flow in vasculatures having higher blood pressure or increased rates of blood flow. The vascular occlusive device may include a support frame for withstanding the higher blood pressure or increased flow rates. The support frame may include a central portion, a distal support arm extending in a distal direction from the ring portion, and a proximal support arm extending in a proximal direction from the ring portion. In one embodiment, the distal support arm may include one or more distal hinges and the proximal support arm may include one or more proximal hinges. In another embodiment, the distal and proximal support arms may each include a helical coil.

### BACKGROUND OF THE INVENTION

Vessel occlusion may be desirable for several reasons. Exemplary circumstances in which vessel occlusion may be desirable include treatment of aneurysms, left atrial appendage, atrial septal defect, fistulas, patent foramen ovale, patent ductus arteriosus, vessel shutdown, or various occlusive purposes in the neuro-vasculature and peripheral vasculature. Vascular occlusion devices may be susceptible to failure in vasculatures having larger sizes (e.g., up to 16-22 millimeters in diameter) with higher blood pressures or increased blood flows.

### SUMMARY OF THE INVENTION

The present invention is generally directed to a vascular plug.

In some example embodiments, the vascular plug comprises a braided mesh portion that expands from a generally linear configuration to a three-dimensional shape. For example, the mesh portion can expand to a generally spherical shape, a concave shape, a flattened oval shape, or a plurality of connected bulbs.

The vascular plug may include a flexible membrane deployed within an interior of the mesh portion when expanded. For example, the flexible membrane may comprise a circular, flat membrane arranged substantially perpendicular to a linear axis of the vascular plug. In another example, the flexible membrane may expand to a position that is non-perpendicular to the axis of the vascular plug.

In some example embodiments, the flexible membrane may be composed of PET, ePTFE, or a thin metallic film.

In some example embodiments, the vascular plug may include an elastic member within the mesh portion to assist in expansion of the vascular plug within a patient.

In some example embodiments, the vascular plug may include a support frame to aid in withstanding higher blood pressures and increased blood flows in larger vasculatures.

In some examples, the support frame may include a central portion for supporting the membrane. In some examples, the central portion may be a substantially circular ring.

In some example embodiments, the support frame may include a distal support arm having at least one hinge when the vascular plug is deployed. In some such example embodiments, the distal support arm may include one or more hinges, such as an upper distal hinge and a lower distal hinge.

In some example embodiments, the support frame may include a proximal support arm having at least one hinge when the vascular plug is deployed. In some such example embodiments, the proximal support arm may include one or more hinges, such as an upper proximal hinge and a lower proximal hinge.

In some example embodiments, the upper distal hinge may be at a higher elevation with respect to the ring portion than the upper proximal hinge and the lower distal hinge may be at a higher elevation with respect to the ring portion than the lower proximal hinge.

In some example embodiments, the hinges may each comprise a curved portion of the respective distal and proximal support arms. Such curved portions may be upward curves or downward curves.

In some example embodiments, the hinges function as joints, dampeners, shock absorbers, springs, articulating regions, regions of increased flexibility, or the like to maintain the structural integrity of the support frame and interconnected flexible membrane under high pressures or increased flow rates.

In some example embodiments, the proximal and distal support arms may extend in opposite directions from a central portion within which the flexible membrane is connected.

In some example embodiments, the support frame, including the central portion, proximal support arm, distal support arm, and hinges may all be formed from a pair of wires.

In some example embodiments, the support frame may include a proximal coil and/or a distal coil which may function as a spring.

The present invention is also directed to a method of deploying a vascular plug having a support frame within a vasculature of a patient.

In some example embodiments, the vascular occlusion device may include two or more membrane supports, each supporting at least one membrane.

In some example embodiments, the vascular occlusion device may include a first membrane and a second membrane.

In some example embodiments, the first and second membranes may be linearly aligned.

In some example embodiments, the first and second membranes may be substantially the same shape and/or dimensions.

In some example embodiments, the first and second membranes may be interconnected by at least one support arm.

In some example embodiments, the at least one support arm linking the first and second membranes may comprise an inverted U-shape.

In some example embodiments, the at least one support arm linking the first and second membranes may comprise a U-shape.

In some example embodiments, the at least one support arm linking the first and second membranes may comprise an S-shape.

In some example embodiments, the at least one support arm linking the first and second membranes may comprise a twinned pair of wires.

In some example embodiments, the vascular occlusion device may include a single lobe or mesh portion having two or more membranes positioned in an interior thereof.

In some example embodiments, the vascular occlusion device may include multiple lobes or mesh portions, each having one or more membranes positioned in an interior thereof.

In some example embodiments, the vascular occlusion device may include a pair of lobes or mesh portions, including a first lobe or mesh portion having a less dense braiding and a second lobe or mesh portion having a more dense braiding.

In some example embodiments, an internal support frame of a vascular occlusion device may include a flexible segment formed from a coil or the like.

### BRIEF DESCRIPTION OF THE DRAWINGS

These and other aspects, features and advantages of which embodiments of the invention are capable of will be apparent and elucidated from the following description of embodiments of the present invention, reference being made to the accompanying drawings, in which

FIG. 1 is an isometric view of a vascular occlusion device according to one embodiment of the present invention.

FIG. 2 is a side view of a vascular occlusion device according to one embodiment of the present invention.

FIG. 3 is an end view of a vascular occlusion device according to one embodiment of the present invention.

FIG. 4 is a side view of a vascular occlusion device attached to a pusher according to one embodiment of the present invention.

FIG. 5 is an isometric view of a support frame for a vascular occlusion device according to one embodiment of the present invention.

FIG. 6 is a side view of a support frame for a vascular occlusion device according to one embodiment of the present invention.

FIG. 7 is a side view of a vascular occlusion device inserted through a vasculature in a compact configuration according to one embodiment of the present invention.

FIG. 8 is a side view of a vascular occlusion device being deployed in a vasculature according to one embodiment of the present invention.

FIG. 9 is a side view of a vascular occlusion device in use in a vasculature according to one embodiment of the present invention.

FIG. 10 is an isometric view of a support frame and membrane for a vascular occlusion device according to one embodiment of the present invention.

FIG. 11 is an isometric view of a support frame for a vascular occlusion device according to one embodiment of the present invention.

FIG. 12 is a side view of a support frame for a vascular occlusion device according to one embodiment of the present invention.

FIG. 13 is an isometric view of a vascular occlusion device according to one embodiment of the present invention.

FIG. 14 is a side view of a vascular occlusion device according to one embodiment of the present invention.

FIG. 15 is a side view of a vascular occlusion device according to one embodiment of the present invention.

FIG. 16 is an upper perspective view of a support frame for a vascular occlusion device according to one embodiment of the present invention.

FIG. 17 is a perspective view of a support frame for a vascular occlusion device according to one embodiment of the present invention.

FIG. 18 is a perspective view of a vascular occlusion device according to one embodiment of the present invention.

FIG. 19 is a side view of a vascular occlusion device according to one embodiment of the present invention.

FIG. 20 is a perspective view of a support frame for a vascular occlusion device according to one embodiment of the present invention.

FIG. 21 is a side view of a vascular occlusion device according to one embodiment of the present invention.

FIG. 22 is a perspective view of a vascular occlusion device according to one embodiment of the present invention.

FIG. 23 is a side view of a vascular occlusion device according to one embodiment of the present invention.

FIG. 24 is a side view of a vascular occlusion device according to one embodiment of the present invention.

FIG. 25 is a side view of a vascular occlusion device according to one embodiment of the present invention.

FIG. 26 is a side view of a vascular occlusion device being deployed in a vasculature according to one embodiment of the present invention.

FIG. 27 is a side view of a vascular occlusion device according to one embodiment of the present invention.

### DETAILED DESCRIPTION

Specific embodiments of the invention will now be described with reference to the accompanying drawings. This invention may, however, be embodied in many different forms and should not be construed as limited to the embodiments set forth herein; rather, these embodiments are provided so that this disclosure will be thorough and complete, and will fully convey the scope of the invention to those skilled in the art. The terminology used in the detailed description of the embodiments illustrated in the accompanying drawings is not intended to be limiting of the invention. In the drawings, like numbers refer to like elements.

Vascular plugs are used for various occlusive purposes in the vasculature. These plugs generally conform to the shape of the blood vessel or blood vessel abnormality thereby occluding and preventing blood flow through or to the target area. Plugs can be used to treat a variety of conditions including aneurysms, left atrial appendage, atrial septal defect, fistulas, patent foramen ovale, patent ductus arteriosus, vessel shutdown, or can be used for various occlusive purposes in the neuro-vasculature and peripheral vasculature.

Vascular plugs generally provide faster occlusion than other occlusive devices such as embolic coils since, rather than filling the target space, the plugs conform to the shape of the target space promoting faster occlusion. Vascular plugs generally are larger than other occlusive devices (such as embolic coils) since they are meant to conform to the target space, rather than fill the target space. This larger profile can make deliverability an issue as compared to other occlusive devices, therefore, vascular plugs need to balance the need for rapid occlusion with the need for ease of deliverability in order to effectively deliver the plug to the target treatment site.

Specific example embodiments are described below. However, it should be understood that any of the features from any of the embodiments can be mixed and matched with each other in any combination. Hence, the present invention should not be restricted to only these embodiments, but any broader combination thereof.

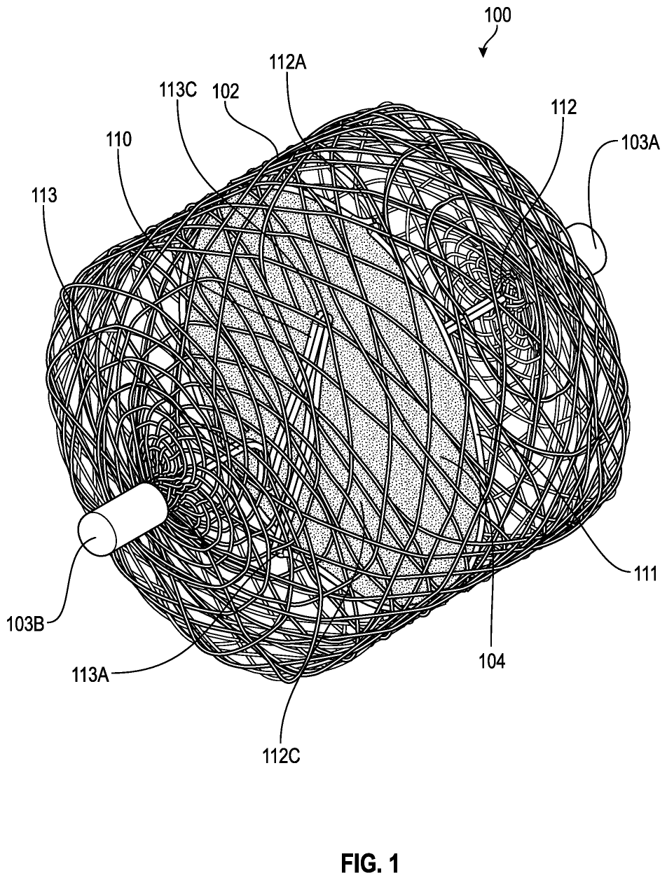

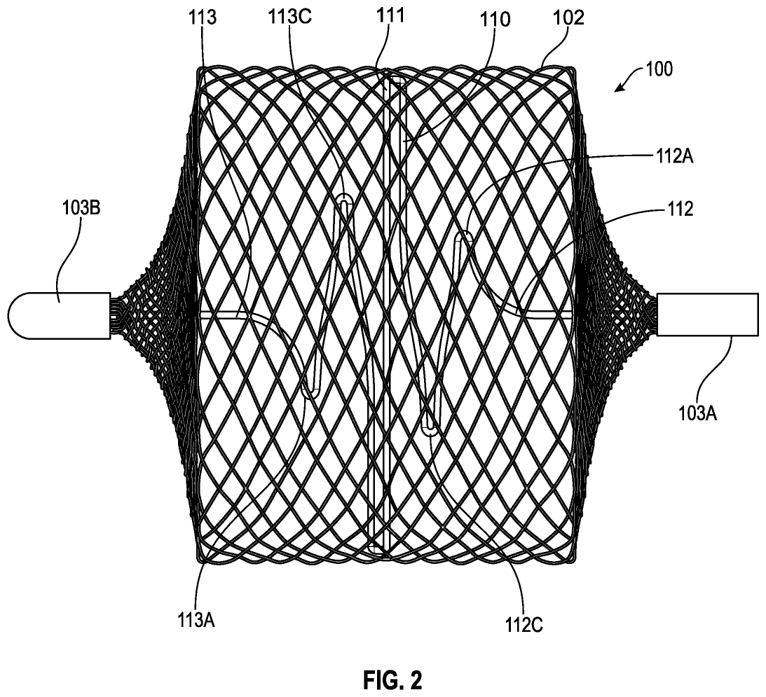

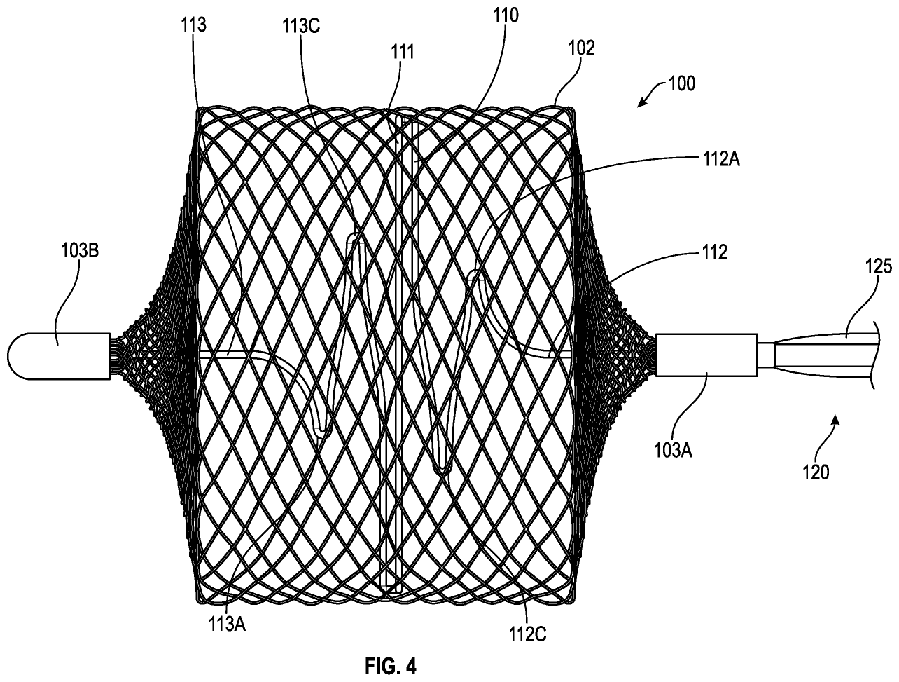

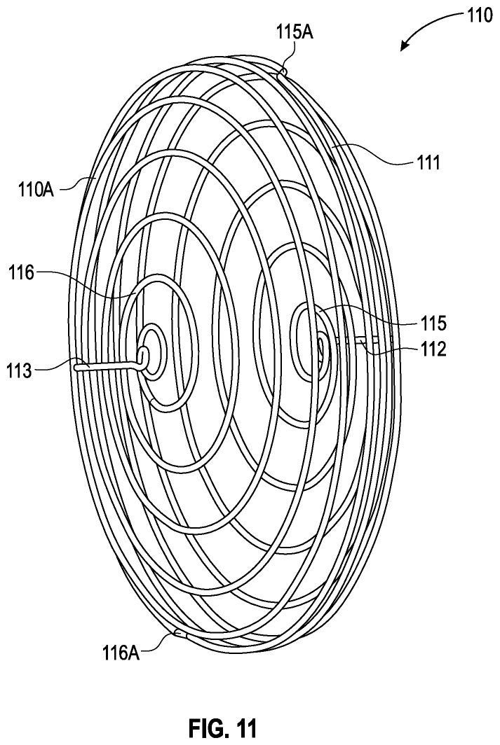

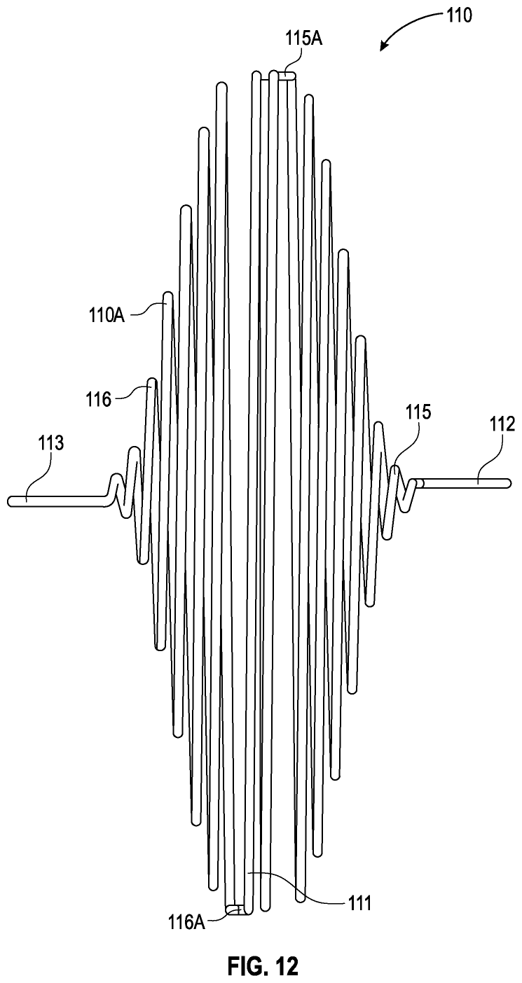

FIGS. 1-12 illustrate various aspects of a vascular plug 100 that may be connected to a distal end of a pusher 120, thereby allowing the plug 100 to be advanced through a catheter 125 to a desired target location in a patient. When a mesh portion 102 of the vascular plug 100 is expanded, a flexible membrane 104 is also expanded within the mesh portion 102 to create a blockage or barrier at the target location. The flexible membrane 104 may function to encourage a thrombogenic response after implantation to aid in occlusion.

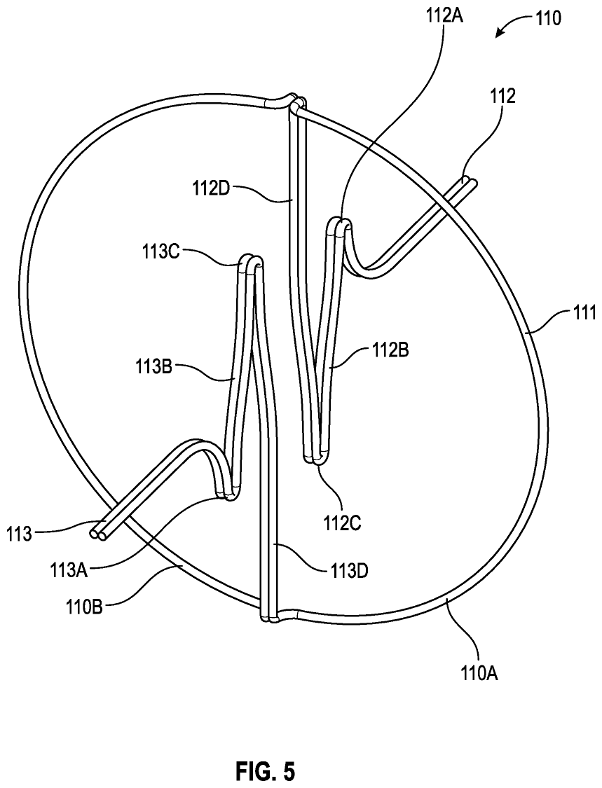

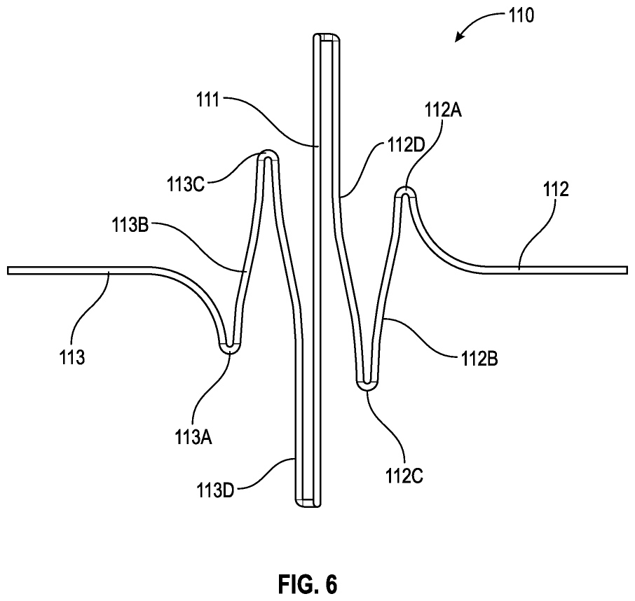

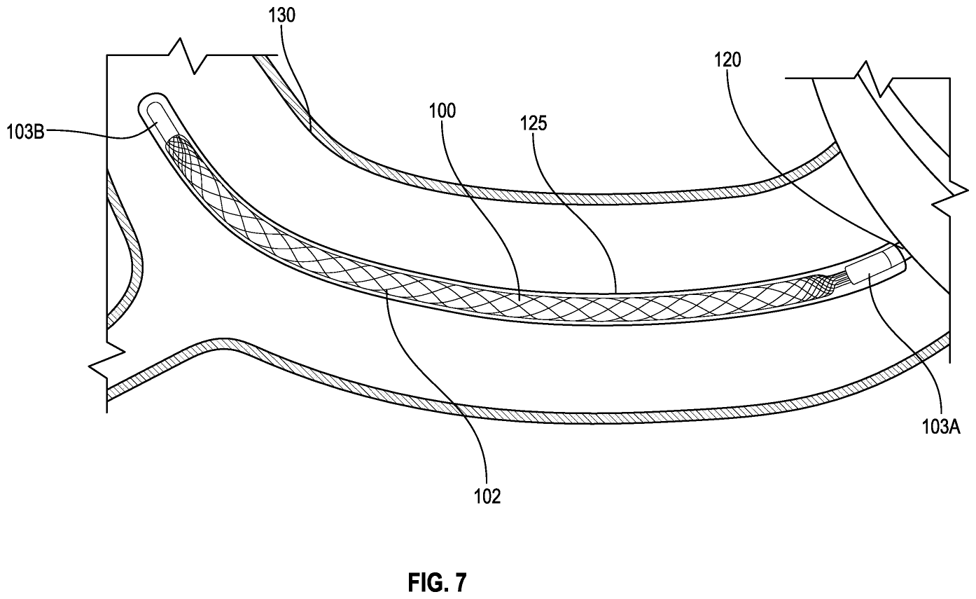

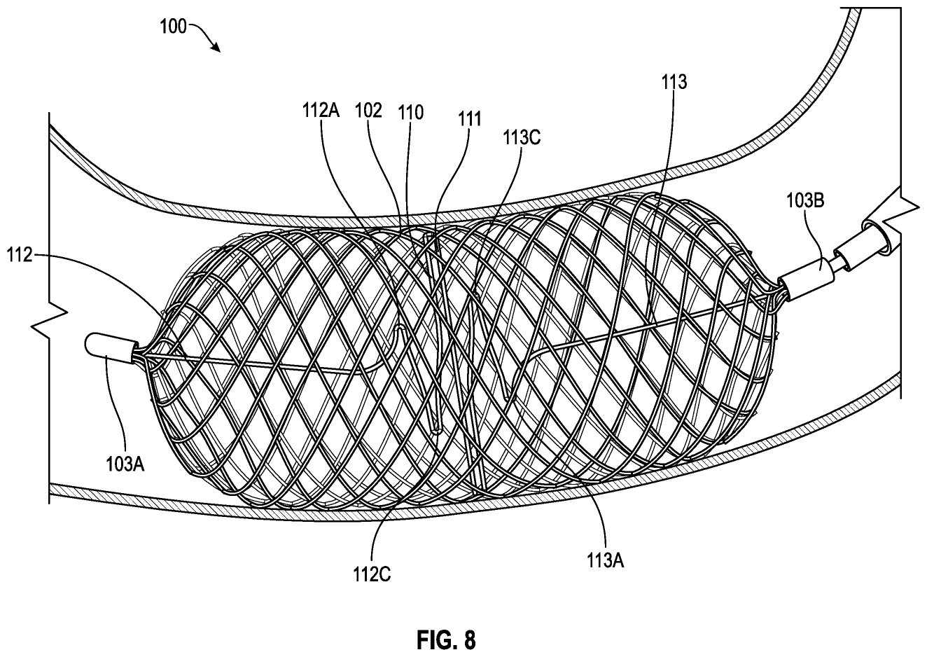

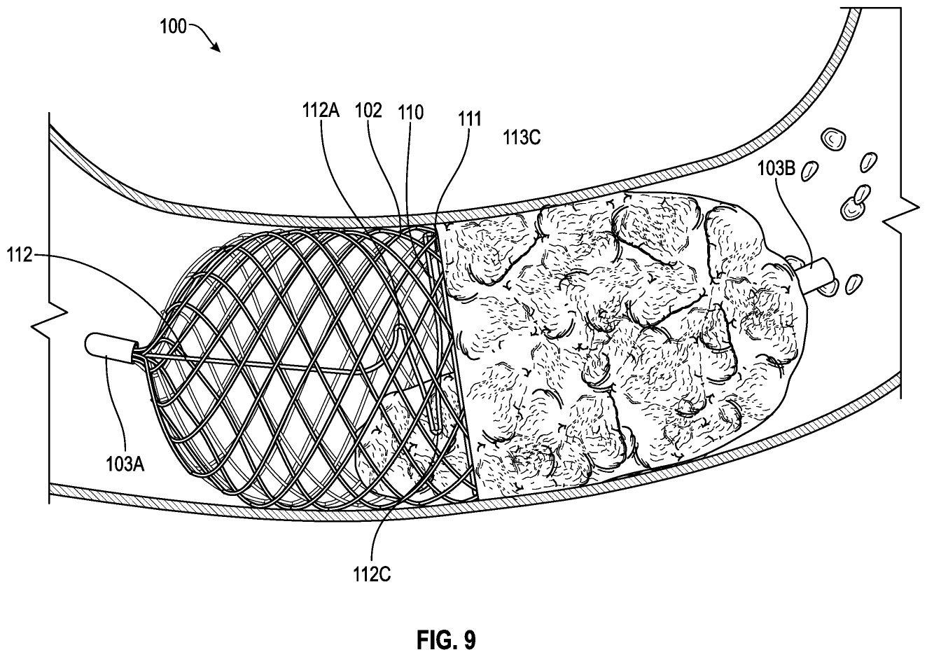

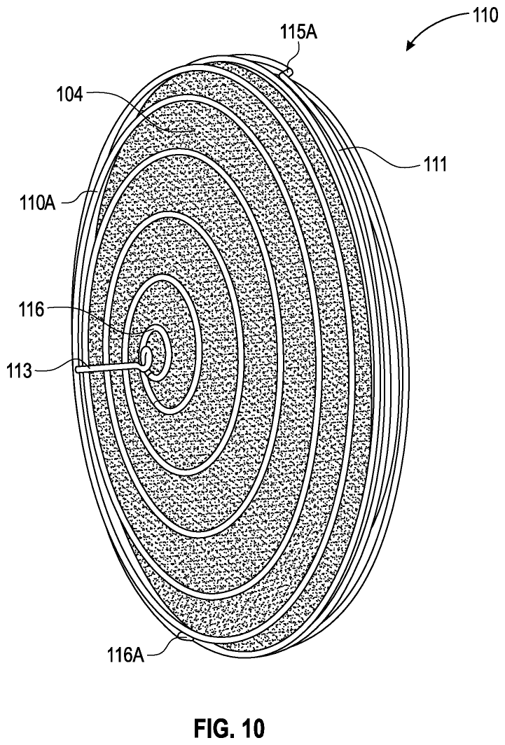

FIGS. 1-4 illustrate an example embodiment of a vascular plug 100 in an expanded configuration for use in occluding a vasculature. FIGS. 5-6 illustrate an example embodiment of a support frame 110 for a vascular plug 100. FIG. 7 illustrates the vascular plug 100 in a compressed, linear configuration within a catheter 125. FIGS. 8-9 illustrate the vascular plug 100 in use in an expanded configuration. FIGS. 10-12 illustrate another example embodiment of a support frame 110 for a vascular plug 100.

The mesh portion 102 may have a radially compressed configuration when constrained in a catheter 125 and a radially expanded configuration when unconstrained. Thus, the mesh portion 102 may expand from an elongated, compressed, cylindrical or linear shape (e.g., when located within the catheter 125) to a longitudinally shorter and expanded shape. The expanded shape may be generally spherical or may take on various other regular or irregular shapes to suit different vasculatures, such as but not limited to a generally cylindrical shape such as shown in the figures. The mesh portion 102 may include an interior space which may expand to a greater volume when the mesh portion 102 expands into the expanded shape.

The wires of the mesh portion 102 can be formed from various materials, such as but not limited to nitinol, cobalt-chromium, stainless steel wires, or combinations thereof. In one example, the mesh portion 102 may be comprised of 48-144 nitinol wires with a diameter range of about 0.0008-0.005 inches. Optionally, one or more radiopaque wires can be used to create the mesh portion 102, to further enhance visualization of the vascular plug 100 during a procedure.

As best shown in FIGS. 1-4, the proximal end of the mesh portion 102 may terminate with a proximal cap member 103A and the distal end of the mesh portion 102 may terminate with a distal cap member 103B. The proximal and distal cap members 103A, 103B may be formed or manufactured in various manners. By way of example, the proximal and distal cap members 103A, 103B may be formed by welding the wires of the mesh portion 102 together, welding the wires to discrete metal caps, crimping metal cap members onto the wires, or using an adhesive to attach discrete caps to the wires. The proximal and distal cap members 103A, 103B in an example embodiment may be composed at least partially of radiopaque materials such that they can be used as visual markers by a physician during a procedure. The proximal cap member 103A may be configured to engage with (e.g., removably connect to or couple with) a pusher 120.

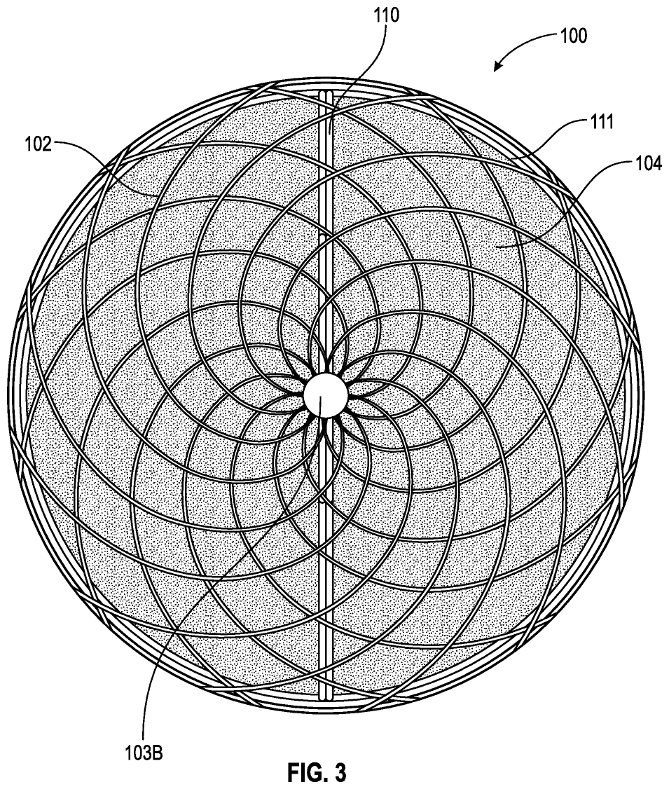

With reference to FIGS. 1 and 3, it can be seen that the vascular plug 100 may include a flexible membrane 104 that is expanded when deployed. The membrane 104 may adopt a radially expanded configuration as the mesh portion 102 adopts a radially expanded configuration, thereby limiting fluid (e.g., blood) passage through the mesh portion 102.

The shape of the membrane 104 upon deployment may vary in different embodiments. In the exemplary embodiment shown in the figures, the flexible membrane 104 may have a circular shape when deployed. However, any other shape capable of occluding a vasculature may be utilized. Generally, the shape of the membrane 104 when deployed and expanded will substantially match the shape of the central portion 111 to which it is attached.

The flexible membrane 104 may be comprised of any material that can be unfolded, straightened, stretched, or otherwise expanded to an enlarged and preferably planar area. The flexible membrane 104 can be comprised of a variety of flexible materials that are biocompatible and that increase a thrombogenic response to aid in forming an occlusion in the patient. For example, polyethylene terephthalate (PET) or expanded polytetrafluoroethylene (ePTFE) can be used. In another example embodiment, a composite of PET and ePTFE can be used. In another example embodiment, the flexible membrane 104 can be composed of a thin-metallic film, such as those created via sputtering or vacuum deposition.

As shown in FIGS. 1-4, the flexible membrane 104 may be supported by a support frame 110. The support frame 110 may be positioned within an interior cavity of the mesh portion 102. As will be described in more detail below, the entire support frame 110 may be formed from a pair of resilient wires 110A, 110B that are adjustable between a compressed, linear configuration (such as for fitting within a catheter 125) and an expanded configuration (such as for expanding within a vasculature). The resilient wires 110A, 110B may comprise various flexible, resilient materials such as but not limited to nitinol. The size (e.g., diameter) of the resilient wires 110A, 110B may vary and, in some embodiments, may be 12 mm or greater. However, in some embodiments, the size of the resilient wires 110A, 110B may be less than 12 mm.

It should be appreciated that the systems and methods shown and/or described herein relating to a support frame 110, 210 for a vascular plug 100, 210 may be utilized with a wide range of different vascular plugs 100, 200 having a wide range of different shapes, orientations, materials, and configurations. As one example, the systems and methods described herein may be utilized with any of the example embodiments of a “Vessel Occluder” shown and described in U.S. Pat. No. 10,470,773. U.S. Pat. No. 10,470,773 is hereby incorporated by reference in its entirety.

FIGS. 5 and 6 illustrate one example embodiment of a support frame 110 for a vascular plug 100. As best shown in FIG. 5, an example embodiment of the support frame 110 may include a central portion 111. The central portion 111 is illustrated as comprising a ring portion that may be circular when expanded, though in some example embodiments the central portion 111 may comprise other shapes (e.g., square-shaped, oval-shaped, etc.) to suit different vasculatures.

The central portion 111 may be oriented such that the plane of the central portion 111 is generally perpendicular to an axis between the proximal and distal ends of the mesh portion 102 (e.g., a longitudinal axis between the proximal and distal cap members 103A, 103B). Such an orientation allows the flexible membrane 104 to be expanded almost completely across the cavity of the mesh portion 102 and thus block passage of fluid from a patient between the proximal and distal ends of the vascular plug 100. It should be appreciated, however, that the flow of fluid across the flexible membrane 104 will in many cases force the flexible membrane 104 slightly off its perpendicular orientation when in use such that the flexible membrane 104 is at a non-perpendicular angle with respect to a longitudinal axis extending through the vascular plug 100.

The central portion 111 may expand to a width or diameter that is similar in size to the largest inner diameter region of the expanded mesh portion 102. In some example embodiments, the central portion 111 may expand to a size that is slightly larger than the inner diameter of the vasculature in which the vascular plug 100 is deployed. Thus, the diameter of the membrane 104 may be oversized compared to the target vessel size. Such a configuration may be desirable as the flow of fluids such as blood across the membrane 104 may cause the central portion 111 to shift off slightly off a perpendicular plane. By ensuring that the central portion 111 is slightly larger than the inner diameter of the vasculature 130 in which the vessel plug 100 is deployed, it can be ensured that the vasculature 130 is fully covered (e.g., without any gaps) even when the central portion 111 is shifted due to such fluid flow against the membrane 104.

The manner by which the flexible membrane 104 is secured to the central portion 111 may vary in different embodiments. In some example embodiments, the flexible membrane 104 may be fixed to the central portion 111 by forming a laminating layer over the flexible membrane 104, around the wire(s) 110A, 110B of the central portion 111, and back upon itself. For example, the flexible membrane 104 may in some embodiments be initially created with PET. A layer of ePTFE may then be disposed or laminated over the PET layer and the central portion 111. Alternatively, the flexible membrane 104 may be stitched to the central portion 111 with metal wires, polymer fibers, or the like. In another example embodiment, various adhesives may be utilized to attach or secure the flexible membrane 104 to the central portion 111. In another example embodiment, the membrane 104 may be bonded to a polymeric sleeve over the central portion 111, made of PET or a heat shrinkable plastic such as cross-linked PET. In another example embodiment, a PET HS tube underneath the membrane 104 may be used to prevent the membrane 104 from sliding around the frame 110 and is bonded to the ePTFE/PET-ePTFE layers. In yet another example embodiment, the flexible membrane 104 may be directly stitched or adhered to the mesh portion 102, such as to the wires forming the mesh portion 102.

In an example embodiment such as shown in FIGS. 5 and 6, the central portion 111 may be supported by a proximal support arm 112 and a distal support arm 113. The proximal support arm 112 may extend in a first (e.g., proximal) direction from the central portion 111 and the distal support arm 113 may extend in a second (e.g., distal), opposite direction from the central portion 111. The first direction may be a proximal direction and the second direction may be a distal direction. As shown in FIGS. 1-4, the proximal support arm 112 may be connected at one of its ends to the proximal cap member 103A. Similarly, the distal support arm 113 may be connected at one of its ends to the distal cap member 103B.

While the figures illustrate a support frame 110 formed of only a single proximal support arm 112 and a single distal support arm 113, it should be appreciated that additional proximal and/or distal support arms 112, 113 may be included in an example embodiment of a support frame 110 to suit different applications. For example, in some example embodiments, the support frame 110 may include two or more proximal support arms 112 and two or more distal support arms 113. In such embodiments, the two or more proximal support arms 112 may converge at the proximal cap member, and the two or more distal support arms 113 may converge at the distal cap member.

As best shown in FIGS. 5 and 6, the proximal support arm 112 may include one or more proximal hinges 112A, 112C linked by one or more ascending or descending linkages 112B, 112D. Such proximal hinges 112A, 112C may function as joints, shock absorbers, springs, resiliency aids, articulating regions, regions of increased flexibility, or other connecting structures which aid in the resiliency of the support frame 110 by absorbing forces from flowing fluids and thereby preventing failure of the vessel plug 100 when under high pressure or flow rate conditions that are common in larger vasculatures.

In one example embodiment, the proximal support arm 112 may include a first proximal hinge 112A and a second proximal hinge 112C. The first proximal hinge 112A may comprise an upper proximal hinge 112A and the second proximal hinge 112C may comprise a lower proximal hinge 112C. Thus, in an example embodiment, the first proximal hinge 112A may comprise a peak, and the second proximal hinge 112C may comprise a trough.

The figures illustrate example embodiments of first and second proximal hinges 112A, 112C which each comprise a rounded curve (e.g., a semi-circular shape). More specifically, it can be seen that the first proximal hinge 112A may comprise a downward curve (also commonly referred to as a concave curve, convex upward curve, or concave downward curve) and that the second proximal hinge 112C may comprise an upward curve (also commonly referred to as a convex curve, convex downward curve, or concave upward curve). However, it should be appreciated that the shape of each of the proximal hinges 112A, 112C may vary in different embodiments. For example, in some embodiments, each of the proximal hinges 112A, 112C may comprise a triangular or square shape.

With reference to FIG. 6, it can be seen that the first and second proximal hinges 112A, 112C may be linked together by a first linkage 112B which extends between and connects the first hinge 112A with the second hinge 112C. The first linkage 112B may thus comprise a descending elongated member connecting the peak of the first proximal hinge 112A with the trough of the second proximal hinge 112C. Similarly, a second linkage 112D may connect the second hinge 112C with the central portion 111. The second linkage 112D may thus comprise an ascending elongated member connecting the trough of the second proximal hinge 112C with the central portion 111.

Continuing to reference FIGS. 5 and 6, it can be seen that the distal support arm 113 may similarly include one or more distal hinges 113A, 113C linked by one or more ascending or descending linkages 113B, 113D. Such distal hinges 113A, 113C may function as joints, shock absorbers, springs, resiliency aids, articulating regions, regions of increased flexibility, or other connecting structures which aid in the resiliency of the support frame 110 and prevent failure of the vessel plug 100 when under high pressure or flow rate conditions that are common in larger vasculatures.

In one example embodiment, the distal support arm 113 may include a first distal hinge 113A and a second distal hinge 113C. The first distal hinge 113A may comprise a lower distal hinge 113A and the second distal hinge 113C may comprise an upper distal hinge 113C. Thus, in an example embodiment, the first distal hinge 113A may comprise a trough, and the second distal hinge 113C may comprise a peak.

The figures illustrate embodiment of first and second distal hinges 113A, 113C which each comprise a rounded curve (e.g., a semi-circular shape). More specifically, it can be seen that the first distal hinge 113A may comprise an upward curve and that the second distal hinge 113C may comprise a downward curve. However, it should be appreciated that the shape of each of the hinges 113A, 113C may vary in different embodiments. For example, in some embodiments, each of the hinges 113A, 113C may comprise a triangular or square shape.

As shown in the figures, the second proximal hinge 112C may be closer to the central portion 111 and membrane 104 than the first proximal hinge 112A. Similarly, the second distal hinge 113C may be closer to the central portion 111 and membrane 104 than the first distal hinge 113A.

As shown in FIG. 6, the first and second distal hinges 113A, 113C of the distal support arm 113 may be linked together by a first linkage 113B which extends between and connects the first distal hinge 113A with the second distal hinge 113C. The first linkage 113B may thus comprise an ascending elongated member connecting the trough of the first distal hinge 113A with the peak of the second distal hinge 113C. Similarly, a second linkage 113D may connect the second distal hinge 113C with the central portion 111. The second linkage 113D may thus comprise a descending elongated member connecting the peak of the second distal hinge 113C with the central portion 111.

The distal support arm 113 may be symmetrical or mirror the proximal support arm 112. However, in some embodiments, the proximal and distal support arms 112, 113 may not be mirror images of each other, or be symmetrical. While an example embodiment is shown and described with specific configurations, orientations, and locations of the respective hinges 112A, 112C, 113A, 113C and linkages 112B, 112D, 113B, 113D of the respective proximal and distal support arms 112, 113, it should be appreciated that various other configurations, orientations, and locations may be utilized in different embodiments. For example, the previously described arrangement of the first proximal hinge 112A and second distal hinge 113C having downward curves or peaks, and the second proximal hinge 112C and second distal hinge 113A having upward curves or troughs, could be reversed in some example embodiments.

It should thus be appreciated that the preceding description, and the accompanying figures, could be inverted from what is described and shown. For example, the first proximal hinge 112A of the proximal support arm 112 may comprise a trough and the second proximal hinge 112C of the proximal support arm 112 may comprise a peak. Similarly, the first distal hinge 113A of the distal support arm 113 may comprise a peak and the second distal hinge 113C of the distal support arm 113 may comprise a trough.

Although the figures illustrate that the proximal support arm 112 includes a pair of proximal hinges 112A, 112C and that the distal support arm 113 includes a pair of distal hinges 113A, 113C, it should be appreciated that one or both of the proximal and/or distal support arms 112, 113 may, in some example embodiments, include additional hinges 112A, 112C, 113A, 113C. For example, the proximal support arm 112 may include three or more proximal hinges 112A, 112C and/or the distal support arm 113 may include three or more distal hinges 113A, 113C. Preferably, each of the proximal and distal support arms 112, 113 will include an even number of hinges 112A, 112C, 113A, 113C. However, in some example embodiments, one or both of the proximal and distal support arms 112, 113 may include only a single hinge 112A, 113A.

With reference to FIG. 6, it can be seen that the first proximal hinge 112A may be at a lower height than the second distal hinge 113C, and that the second proximal hinge 112C may be at a lower height than the first distal hinge 113A. However, various other configurations may be utilized in different embodiments. For example, the reverse configuration could be utilized, in which the first proximal hinge 112A is at a greater height than the second distal hinge 113C and the second proximal hinge 112C is at a greater height than the first distal hinge 113A. In another example embodiment, the first proximal hinge 112A may be at the same height as the second distal hinge 113C and the second proximal hinge 112C may be at the same height as the first distal hinge 113A.

Continuing to reference FIG. 6, it can be seen that each of the proximal and distal linkages 112B, 112D, 113B, 113D may extend at an angular orientation. More specifically, it can be seen that the first proximal linkage 112B may be angled or curved towards the second proximal linkage 112D, the second proximal linkage 112D may be angled or curved towards the first proximal linkage 112B, the first distal linkage 113B may be angled or curved towards the second distal linkage 113D, and the second distal linkage 113D may be angled or curved towards the first distal linkage 113B. In some embodiments, one or more of the linkages 112B, 112D, 113B, 113D may instead extend linearly (e.g., vertically), rather than at an angle.

With reference to the angles of each of the hinges 112A, 112C, 113A, 113C, and the angles between the respective linkages 112B, 112D, 113B, 113D, it should be appreciated that such angles may vary in different embodiments. In the exemplary embodiments shown in the figures, each of these angles are shown as comprising an acute angle. However, such angles may be greater or lesser than is shown in the example embodiments of the figures. For example, the angle between the first and second proximal linkages 112B, 112D on the one hand and/or the angle between the first and second distal linkages 113B, 113D may be greater or lesser than is shown in FIG. 6.

As best shown in FIG. 5, the entire support frame 110 may be formed from a pair of wires 110A, 110B which may be secured against each other by various methods. More specifically, it can be seen that a first wire 110A and a second wire 110B may be fused together side-by-side to form the proximal support arm 112, including the proximal hinges 112A, 112C and proximal linkages 112B, 112D. The wires 110A, 110B may then separate from each other to form the central portion 111, and then fuse back together to form the distal support arm 113, including the distal hinges 113A, 113C and distal linkages 113B, 113D. In such an embodiment, the entirety of the support frame 110, including the support arms 112, 113, hinges 112A, 112C, 113A, 113C, and linkages 112B, 112D, 113B, 113D, may be integrally formed from the pair of wires 110A, 110B.

FIGS. 7-9 illustrate an example embodiment of a vascular plug 100 in use to occlude a vasculature 130. In operation, the catheter 125, with the pusher 120 inside of it, may be advanced within a vasculature 130 (e.g., a vessel or lumen) of a patient until the distal end of the catheter 125 is adjacent to the target occlusion site such as shown in FIG. 7. By way of example, the distal end of the catheter 125 may be positioned within or at the mouth of an aneurysm.

As shown in FIG. 7, vascular plug 100 may be delivered to the vasculature 130 in a compressed, compact, linear, cylindrical configuration. In an example embodiment, the vascular plug 100 may be positioned within a catheter 125 for delivery; the catheter 125 being advanced through the vasculature 130 by an elongated pusher 120. Various types of catheters 125 may be utilized for delivery of the vascular plug 100. By way of non-limiting example, TERUMO GLIDECATH 4F, 5F, and 5F XP catheters may be utilized for delivery of the vascular plug 100 in some embodiments.

After arrival at the occlusion site, the pusher 120 may be distally advanced (or optionally the catheter 125 may be retracted) such that the vascular plug 100 is exposed at a distal end of the catheter 125 and located at the desired occlusion site (e.g., within an aneurysm or within a blood vessel). As the vascular plug 100 is exposed, the mesh portion 102 and the flexible membrane 104 may expand to substantially block flow of bodily fluid such as blood past it.

FIG. 8 illustrates an example embodiment of an expanded vascular plug 100 having been delivered to an occlusion site prior to detachment of the pusher 120. As illustrated, the pusher 120 may be removably connected to the proximal end of the vascular plug 100, e.g., to the proximal cap member 103A. As shown in FIG. 8, the vascular plug 100, upon advancement of the pusher 120 or retraction of the catheter 125, may expand into its expanded configuration. The expanded vascular plug 100 will generally be positioned so as to substantially cover the interior diameter of the vasculature 130 at the occlusion site.

After delivery of the vascular plug 100 to the occlusion site, the pusher 120 may be detached from the vascular plug 100 and withdrawn from the vasculature 130. The manner by which the pusher 120 is detached may vary in different embodiments. By way of example, detachment systems such as those shown and described in U.S. Pat. No. 8,182,506, US20060200192, US20100268204, US20110301686, US20150289879, US20151073772, and US20150173773, all of which are incorporated by reference in their entireties, may be utilized.

However, in some example embodiments, the vascular plug 100 may instead be used only temporarily. In such example embodiments, the vascular plug 100 may be deployed within the vasculature 130 temporarily, and then later withdrawn back into the catheter 125. For example, it may be beneficial to use the vascular plug 100 to temporarily occlude a space within a patient's vasculature during an endovascular procedure (e.g., while implanting coils, while implanting a liquid embolic, etc.).

FIG. 9 illustrates a deployed, expanded vascular plug 100 that has been detached from a pusher 120, the pusher 120 not being visible as it has previously been withdrawn. As shown in FIG. 9, body fluid such as blood may form a thrombosis both on the membrane 104 and the mesh portion 102. The structure and shape of the support frame 110, including the hinges 112A, 112C, 113A, 113C, functions to keep the vascular plug 100 in place with the membrane 104 covering the internal diameter of the vasculature 130 even in larger diameter (e.g., 8-22 millimeters) vasculatures 130 susceptible to higher pressures and/or increased rates of flow.

FIGS. 10-12 illustrate an additional example embodiment of a support frame 110 for a vascular plug 100. In such an example embodiment, it can be seen that the proximal support arm 112 may include a proximal coil 115 and the distal support arm 113 may include a distal coil 116. However, it should be appreciated that, in some embodiments, the vascular plug 100 may only include one of a proximal coil 115 or distal coil 116. For example, in some embodiments, only the proximal support arm 112 may include a proximal coil 115 while the distal support arm 113 does not include a distal coil 116, or vice versa.

In the example embodiment shown in FIGS. 10-12, it can be seen that an example embodiment of a vascular plug 100 may comprise a support frame 110 including a central portion 111 in or to which the membrane 104 may be secured. The central portion 111 may form a circular structure such as a ring or may be comprised of various other shapes as previously discussed. Although not shown, it should be appreciated that the support frame 110 illustrated in FIGS. 10-12 may be positioned within the interior space of a mesh portion 102 as previously discussed.

Continuing to reference FIGS. 10-12, a proximal coil 115 may be formed in a proximal direction from the central portion 111 and a distal coil 116 may be formed in a distal direction from the central portion 111. The proximal and distal coils 115, 116 may function, either separately or collectively, as a spring to absorb or resist forces from fluid flow and thus maintain an optimal position and orientation (e.g., perpendicular or slightly off-axis) of the membrane 104 for occlusion of the vasculature 130.

In the illustrated embodiment, the windings of the proximal coil 115 may decrease in diameter in the proximal direction from the central portion 111. The windings of the distal coil 116 may decrease in diameter in the distal direction from the central portion 111. The proximal and distal coils 115, 116 may be connected to each other at their respective widest segments.

In an example embodiment, the proximal and distal coils 115, 116 may be mirror images or substantially similar to each other. In other embodiments, the proximal and distal coils 115, 116 may not be mirror images or substantially similar. In such embodiments, the respective widest and narrowest diameters, number of windings, rate of descending widths, effective lengths, and/or various other properties of the respective proximal and distal coils 115, 116 may differ from each other.

The proximal coil 115 and the distal coil 116 may each comprise one or more wires 110A. In the illustrated embodiment shown in FIGS. 10-12, a single wire 110A may form both the proximal and distal coils 115, 116. In such an embodiment, the single wire 110A may be heat set into a secondary shape comprised of a proximal support arm 112, a proximal coil 115 formed from helical windings of ascending diameter, a distal coil 116 form from helical windings of descending diameter, and a distal support arm 113. In other embodiments, two or more wires 110A, 110B may be connected together and separately or together heat set into the desired helical coil shape.

The central portion 111 of the support frame 110 of the vascular plug 100 may comprise a circular or ovular ring from which the proximal coil 115 extends in the proximal direction and the distal coil 116 extends in the distal direction. In the example embodiment shown in FIGS. 10-12, the central portion 111 comprises a pair of identical, concentric rings that are spaced apart from each other. The membrane 104 may be secured to the circular or ovular ring of the central portion 111, or between a pair of identical, concentric rings, using various methods such as those previously discussed herein. In an example embodiment, the distance between the pair of rings forming the central portion 111 may be substantially similar to the width of the membrane 104 such that the membrane 104 may be sandwiched between the pair of rings forming the central portion 111.

With reference to FIGS. 10-12, it can be seen that the widest winding of the proximal coil 115 may be connected to the first ring of the central portion 111 by a proximal connector 115A and that the widest winding of the distal coil 116 may be connected to the second ring of the central portion 111 by a distal connector 116A. The proximal and distal connectors may be integrally formed from the same single wire 110A that forms the proximal and distal coils 115, 116, or may be comprised of separate wire segments.

The proximal and distal connectors 115, 116 may extend at various angles. Although not shown, in some example embodiments, the proximal and distal connectors 115A, 116A may extend at a right angle between the respective proximal/distal coils 115, 116 and the central portion 111. The figures illustrate an embodiment in which the proximal connector 115A is positioned at the top and the distal connector 116A is positioned at the bottom of the support frame 110 of the vascular plug 100. However, it should be appreciated that the proximal and distal connectors 115A, 116A may be positioned at various other locations along the radial circumference and thus should not be construed as limited to the particular configuration shown in the example embodiment illustrated in the figures.

The manner by which the support frame 110 may be fabricated may vary in different embodiments. In one example embodiment, the support frame 110 may be heat set into a desired configuration such that the support frame 110 will naturally form into the desired configuration when unconstrained. As a non-limiting example, the embodiment shown in FIGS. 10-12 may be fabricated using a fixture such as a mandrel which is tapered so as to heat set one or more wires 110A, 110B into the desired helical coil shapes shown in the figures.

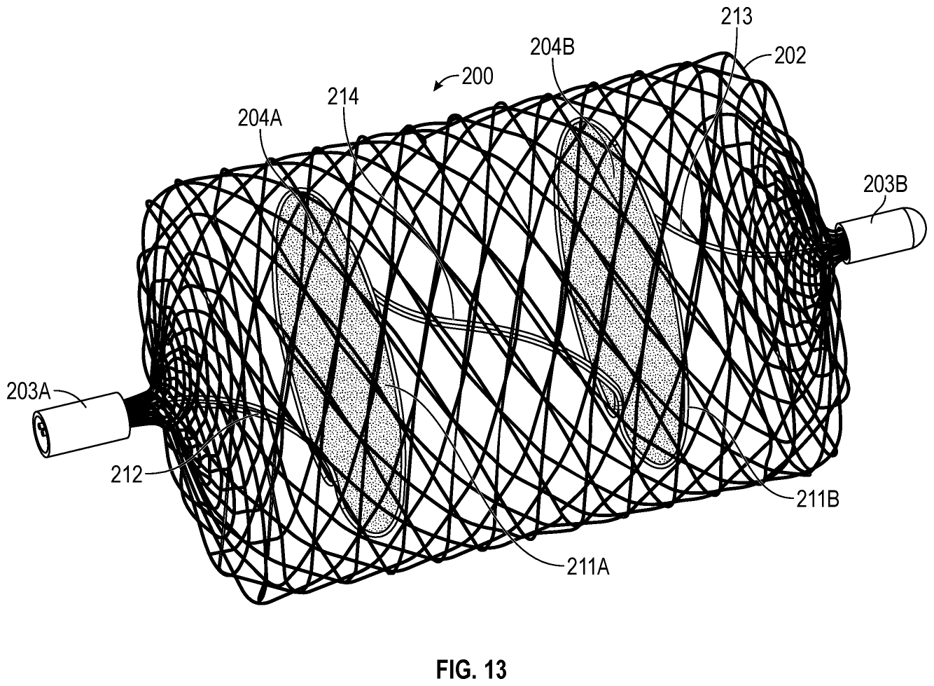

FIGS. 13-23 illustrate additional example embodiments of a vascular plug 200 which may utilize multiple membranes 204A, 204B. It should be appreciated that any and all of the previous features or characteristics discussed above with respect to FIGS. 1-12, such as but not limited to membrane formation, composition, orientation, positioning, and sizing, may equally apply to the additional embodiments shown in FIGS. 13-23 and discussed below. Conversely, it should also be appreciated that any and all of the features or characteristics discussed below with respect to FIGS. 13-23 may equally apply to the previous embodiments shown in FIGS. 1-12 and discussed above.

The use of multiple membranes may provide improved occlusion performance in various peripheral embolization products while minimizing the potential impact to overall device length and tracking performance. FIGS. 13-15, 18-19, and 24 illustrate example embodiments of a single lobe vascular plug 200 having multiple membranes 204A, 204B within a single mesh portion 202. FIGS. 21-23 illustrate example embodiments of a dual lobe vascular plug 250 having a pair of distinct mesh portions 250A, 250B each having its own internal membrane 254A, 254B.

FIGS. 13-15 illustrate an example embodiment of a vascular plug 200. The vascular plug 200 may include a pair of membranes 204A, 204B which may be deployed within a mesh portion 202. While only two membranes 204A, 204B are shown in the example embodiment illustrated in FIG. 13, it should be appreciated that more than two membranes, such as three or more membranes, may be utilized in any of the example embodiments of vascular plugs 100, 200 shown and/or described herein.

Continuing to reference FIGS. 13-15, it can be seen that the vascular plug 200 may comprise a mesh portion 202. The mesh portion 202 may be adjustable between at least two configurations. For example, the mesh portion 202 may be adjustable between a collapsed configuration, such as for use during delivery and/or retrieval, and an expanded configuration, such as for use during deployment in the body. While FIG. 13 illustrates a substantially tubular, cylindrical shape for the mesh portion 202, it should be appreciated that the mesh portion 202 may comprise various other shapes when in its expanded configuration.

The mesh portion 202 may include an internal space, with a volume of the internal space being larger in the expanded configuration than in the collapsed configuration. A support frame 210 may be positioned within the internal space of the mesh portion 202, with the support frame 210 similarly being adjustable between a collapsed configuration and an expanded configuration. The support frame 210 may comprise one or more membrane supports 211A, 211B, each supporting one or more membranes 204A, 204B. By way of example, the support frame 210 may comprise a first membrane support 204A supporting a first membrane 204A and a second membrane support 204B supporting a second membrane 204B.

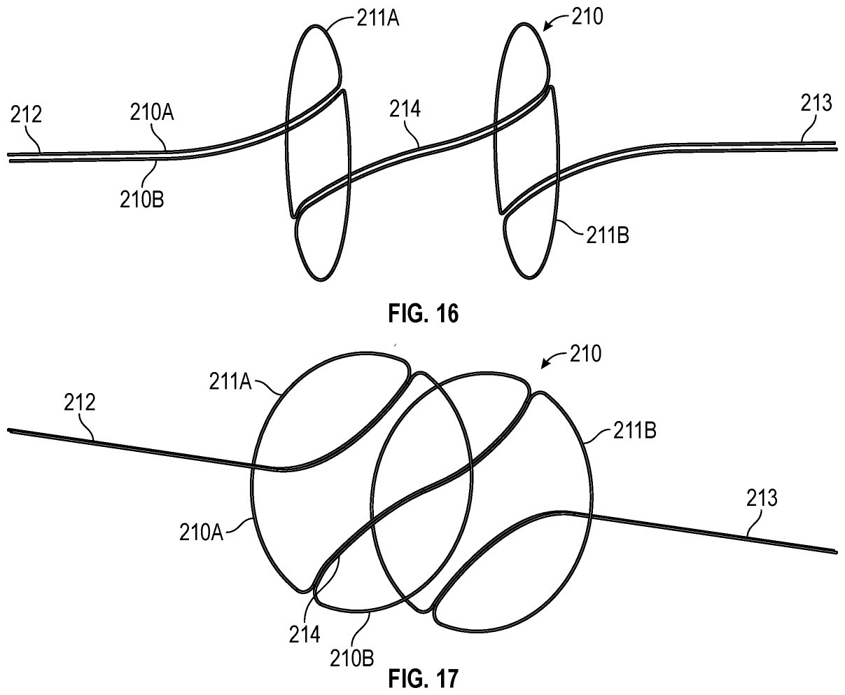

FIGS. 16-17 illustrate an example embodiment of a support frame 210 for a vascular plug 200. As shown, the support frame 210 may comprise one or more support arms 212, 213, 214. A first (proximal) support arm 212 may be sized and shaped to fit within a proximal region of the interior of the mesh portion 202. A second (distal) support arm 213 may be sized and shaped to fit within a distal region of the interior of the mesh portion 202. A third (medial) support arm 214 may be sized and shaped to fit within a medial region of the interior of the mesh portion 202, between the first and second support arms 212, 213. The third support arm 214 may comprise a substantially S-shaped configuration.

Continuing to reference FIGS. 16-17, it can be seen that the support frame 210 may include one or more membrane supports 211A, 211B, each being configured to support one or more membranes 204A, 204B therein. In the illustrated example embodiments, a first membrane support 211A may support a first membrane 204A and a second membrane support 211B may support a second membrane 204B. However, one or more of the membrane supports 211A, 211B may, in some embodiments, support two or more such membranes 204A, 204B.

The shape, size, orientation, and location of the membrane supports 211A, 211B may vary in different embodiments, and thus should not be construed as limited in scope by the example embodiments shown in the figures. In an example embodiment, the membrane supports 211A, 211B may comprise substantially the same shape and/or substantially the same dimensions. In other example embodiments, one or more of the membrane supports 211A, 211B may have a different shape and/or dimensions than one or more of the remaining membrane supports 211A, 211B.

In the example embodiment shown in FIGS. 16-17, it can be seen that the membrane supports 211A, 211B may comprise a substantially circular or ring shape. The shape of the membrane supports 211A, 211B may be similar to that of the cross-section of the mesh portion 202. While the figures illustrate that the membrane 204A, 204B is substantially the same shape as the membrane support 211A, 211B to which it is attached, it should be appreciated that the membrane 204A, 204B may be a different shape than its associated membrane support 211A, 211B in some example embodiments. Generally, it should be appreciated that the membrane supports 211A, 211B of the example embodiment shown in FIGS. 16-17 may have the same characteristics as the central portion 111 shown in, e.g., FIGS. 5-6.

As shown in FIGS. 13-17, the support frame 210 may comprise a proximal support arm 212, a first membrane support 211A, a medial support arm 214, a second membrane support 211B, and a distal support arm 213. However, it should be appreciated that more or less support arms 212, 213, 214 may be utilized in different embodiments. Further, more or less membrane supports 211A, 211B may be utilized in different embodiments as previously discussed herein.

The proximal support arm 212 may extend at least partially through a proximal segment of the interior of the mesh portion 202. As best shown in FIGS. 13-15, a first or proximal end of the proximal support arm 212 may be secured within a proximal cap 203A. A second or distal end of the mesh portion 202 may be attached or integral with the first membrane support 211A. Thus, the proximal support arm 212 may extend between a proximal end of the mesh portion 202 and the first membrane support 211A.

The distal support arm 213 may extend at least partially through a distal segment of the interior of the mesh portion 202. As best shown in FIGS. 13-15, a first or proximal end of the distal support arm 213 may be attached or integral with the second membrane support 211B. A second or distal end of the distal support arm 213 may be secured within a distal cap 203B. Thus, the distal support arm 213 may extend between a distal end of the mesh portion 202 and the second membrane support 211B.

The medial support arm 214 may extend through a medial segment of the interior of the mesh portion 202. The medial support arm 214 may function to interconnect the first and second membrane supports 211A, 211B such as shown in FIGS. 13-17. In the illustrated example embodiment, it can be seen that the medial support arm 214 may extend in a substantially diagonal orientation, such as between an upper end of the first membrane support 211A and a lower end of the second membrane support 211B, or vice versa. However, in some example embodiments, the medial support arm 214 may instead extend linearly, such as parallel to a longitudinal axis extending through a center of the mesh portion 202. The medial support arm 214 may also extend in various other orientations, such as those discussed in more detail below.

With reference to FIGS. 13-15, it can be seen that, in an example embodiment, each of the proximal and distal support arms 212, 213 may extend in an arc along a substantially diagonal path. For example, the proximal support arm 212 may extend in an arc downwardly between the proximal cap 203A and a lower end of the first membrane support 211A. Conversely, the distal support arm 213 may extend in an arc downwardly between the upper end of the second membrane support 211B and the distal cap 203B. However, various other orientations may be utilized for one or both of the proximal and distal support arms 212, 213 in different example embodiments.

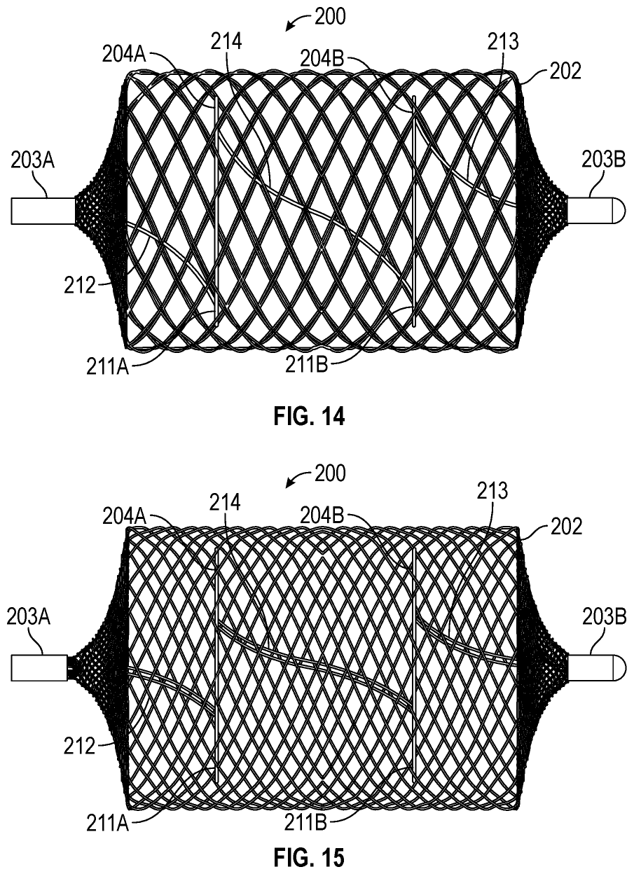

The number of wires forming the support frame 210 may vary in different embodiments. FIG. 14 illustrates an example embodiment in which the support frame 210 is formed from a single wire 210A, which may be heat set to expand into the illustrated, expanded configuration when unconstrained. FIGS. 15-17 illustrate an example embodiment in which the support frame 210 may be formed from a pair of wires 210A, 210B. In such example embodiments, a first wire 210A and a second wire 210B may extend near each other to form the support arms 212, 213, 214, and diverge to form the membrane supports 211A, 211B.

FIG. 16 illustrates an embodiment in which the wires 210A, 210B are slightly spaced apart from each other for the support arm 212, 213, 214 segments of the support frame 210. FIG. 17 illustrates an embodiment in which the wires 210A, 210B are secured against each other form a twinned pair of wires 210A, 210B for the support arm 212, 213, 214 segments of the support frame 210. In some embodiments, three or more wires 210A, 210B may be used to form all or parts of the support frame 210.

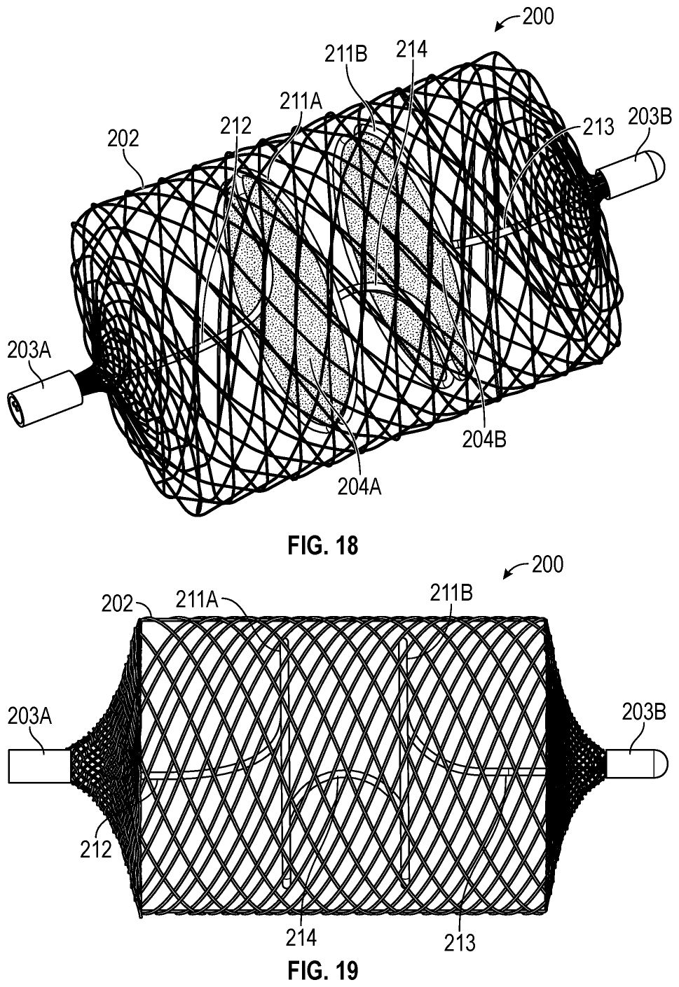

FIGS. 18-20 illustrate an example embodiment of a vascular plug 200 in which the support frame 210 has been slightly altered when compared with the previously discussed example embodiment. It can be seen that, in such an example embodiment, the proximal and distal support arms 212, 213 may extend substantially linearly across the respective proximal and distal segments of the interior of the mesh portion 202, with a steeper incline just prior to transitioning into or connecting to the membrane supports 211A, 211B.

It can also be seen that the medial support arm 214 may form an inverted U-shape configuration, with a first end of the medial support arm 214 being integral with or attached to a lower end of the first membrane support 211A and a second end of the medial support arm 214 being integral with or attached to a lower end of the second membrane support 211B. Although not illustrated, the inverse configuration may also be utilized, with the medial support arm 214 instead forming a U-shape configuration, with the first end of the medial support arm 214 being integral with or attached to an upper end of the first membrane support 211B and the second end of the medial support arm 214 being integral with or attached to an upper end of the second membrane support 211B.

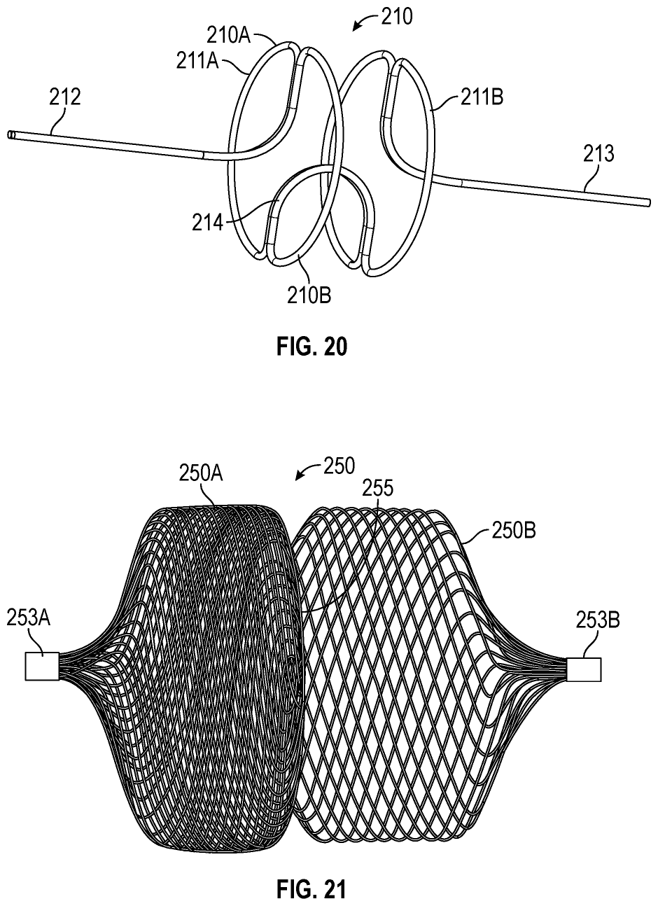

FIGS. 18-19 illustrate an example embodiment in which the support frame 210 may be formed from a single wire. FIG. 20 illustrates an example embodiment in which the support frame 210 may be formed from a twinned pair of wires 210A, 210B. As shown in FIG. 20, the twinned pair of wires 210A, 210B may be cinched or secured together by cinches, clamps, bands, or the like. It should be appreciated that the same configuration may be utilized for any of the other embodiments shown and/or described herein. It should also be appreciated that one or more of the cinches, clamps, bands, or the like may be formed from a radiopaque material so as to form a radiopaque marker for visualization purposes.

FIGS. 21-23 and 25-27 illustrate example embodiments of a multi-lobe vascular plug 250, which may include a first mesh portion 250A and a second mesh portion 250B, with each of the mesh portions 250A, 250B having an interior in which a membrane 254A, 254B may be positioned. In the illustrated embodiments, it can be seen that the first mesh portion 250A may have a first membrane 254A and that the second mesh portion 250B may have a second membrane 254B.

The use of multiple lobes in a vascular plug 250 may provide benefits over a single-lobe design. One lobe may offer radial force and stability, while the other lobe may offer a denser braid. The use of additional lobes may allow the device to be tailored to certain procedures, such as the use of a first lobe to enter the saccular space of an aneurysm and any additional lobes to be deployed in the inflow or outflow vessel. The use of variable stiffness of the lobes (e.g., wherein each of the lobes is a different stiffness) may allow for optimization with trackability, lower retraction forces, minimal “jumping” during delivery, and the opportunity to reduce the overall profile of the device.

An internal membrane may be placed inside one or both lobes to increase occlusion efficacy. Any length mismatch between collapsed internal membranes and collapsed outer braid structures may be addressed by, e.g., one or both ends of the internal wireframe utilizing a flexible spring or laser-cut hypotube to allow for length matching. Such a configuration may also allow for more flexibility when tracking through tortuosity.

FIG. 21 illustrates an example embodiment of a vascular plug 250 having a dual lobe, single membrane design. As shown in FIG. 21, the vascular plug 250 may comprise a first mesh portion 250A and a second mesh portion 250B. The first and second mesh portions 250A, 250B may be interconnected to each other and separated by an interconnection area 255 having a reduced diameter when compared to the mesh portions 250A, 250B themselves. Although not shown, one or more membranes may be positioned within one or both mesh portions 250A, 250B, and/or within the interconnection area 255 between the two mesh portions 250A, 250B. The first mesh portion 250A may terminate proximally in a proximal cap 253A and the second mesh portion 250B may terminate distally in a distal cap 253B.

It should be appreciated that the braiding density, or picks per inch, of each mesh portion 250A, 250B may be different. Thus, the first (proximal) mesh portion 250A may have a higher braiding density than the second (distal) mesh portion 250B, or vice versa. In this manner, the less dense mesh portion 250A may provide more radial force for stability, and the more dense mesh portion 250B may provide lower radial force by including more wires and/or smaller diameter wires in the braid.

The manner by which the two lobes, or two mesh portions 250A, 250B are interconnected to each other may vary in different embodiments. Generally, the interconnection area 255 between the two mesh portions 250A, 250B may include a joint. In an example embodiment, a lumen may be laser welded to the interconnection area 255 to link the two mesh portions 250A, 250B together while maintaining a passage therebetween.

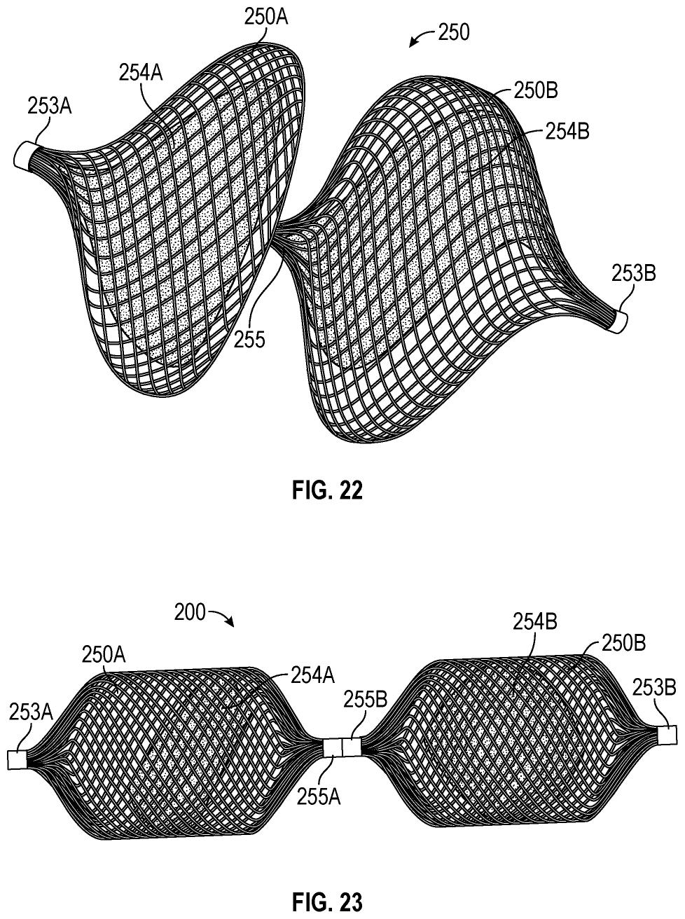

FIG. 22 illustrates an example embodiment of a vascular plug 250 having a dual lobe, dual membrane design. As shown in FIG. 22, the vascular plug 250 may comprise a first mesh portion 250A having a first membrane 254A and a second mesh portion 250B having a second membrane 254B. The first and second mesh portions 250A, 250B may be interconnected to each other and separated by an interconnection area 255 having a reduced diameter when compared to the mesh portions 250A, 250B themselves. The first mesh portion 250A may terminate proximally in a proximal cap 253A and the second mesh portion 250B may terminate distally in a distal cap 253B.

As with the previous embodiment, the first mesh portion 250A may have a different density than the second mesh portion 250B. Additionally, the dimensions of the respective mesh portions 250A, 250B and/or membranes 254A, 254B may be different from each other (e.g., the first mesh portion 250A may have a different diameter or length than the second mesh portion 250B).

FIG. 23 illustrates another example embodiment of a vascular plug 250 having a dual lobe, dual membrane design. In the illustrated embodiment, it can be seen that the interconnection area 255 between the two mesh portions 250A, 250B may include one or more connectors 255A, 255B, such as cinches, clamps, bands, joints, or the like, to interconnect the two mesh portions 250A, 250B with each other.

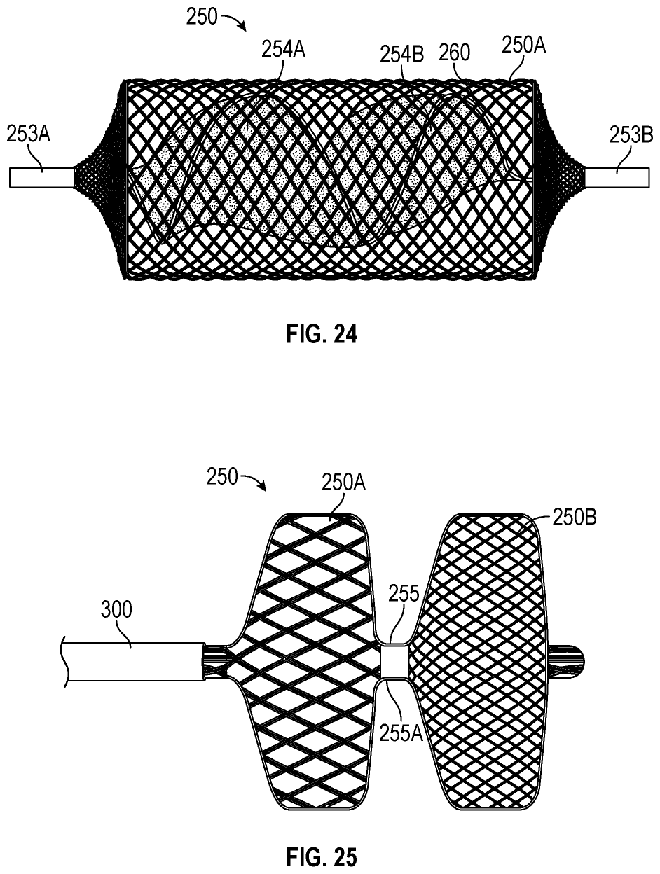

FIG. 24 illustrates an example embodiment of a vascular plug 250 having a single lobe, dual membrane design. As shown in FIG. 24, a single tubular mesh portion 250A may include an interior space within which is positioned a helical support frame 260 which supports a pair of membranes 254A, 254B, or a single membrane 254A which extends along the length of the helical frame 260.

FIG. 25 illustrates an example embodiment of a vascular plug 250 exiting a delivery device 300, with the vascular plug 250 having a first mesh portion 250A comprising a looser (less dense) braid pattern and a second mesh portion 250B comprising a denser braid pattern. The interconnection area between the mesh portions 250A, 250B may include a connector 255A, which may be radiopaque for visualization purposes in some embodiments.

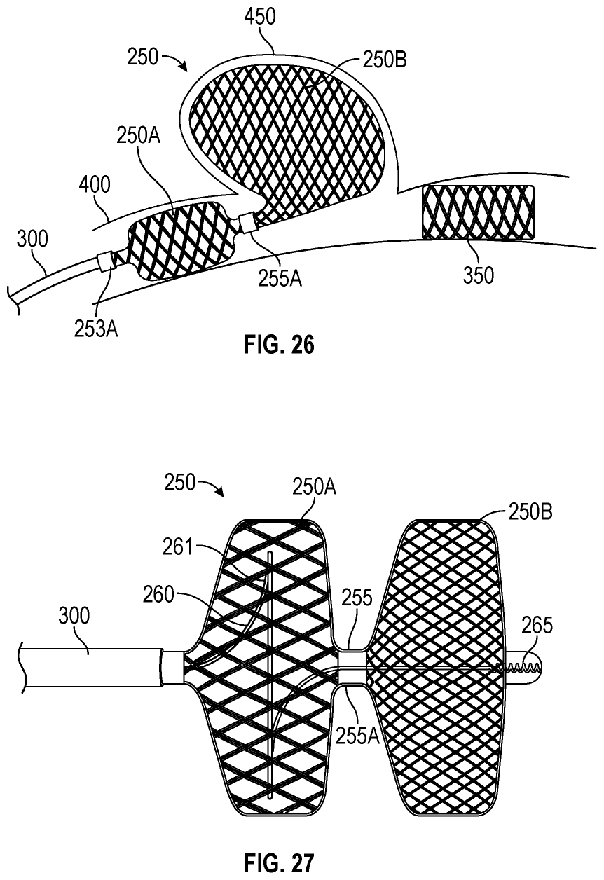

FIG. 26 illustrates the usage of a multiple lobe vascular plug 250 for treatment of a saccular aneurysm 450 in a vessel 400. As illustrated, a first mesh portion 250A may be positioned within the vessel 400 adjacent to the aneurysm 450 and a second mesh portion 250B may be positioned within the aneurysm 450 itself. A separate vascular plug 350 may also be deployed in the inflow or outflow vessel 450.

FIG. 27 illustrates a multiple lobe vascular plug 250 including a single membrane. As shown in FIG. 27, a support frame 260 may extend across and between the two mesh portions 250A, 250B, with the membrane support 261 being positioned in the first mesh portion 250A. The support frame 260 may include a flexible segment 265 to aid in even and uniform collapse of inner and outer structures, even when each of the mesh portions 250A, 250B may have different properties (e.g., densities or dimensions). The flexible segment 265 may comprise a coil or the like and may be integral with the support frame 260 or attached thereto. It should be appreciated that, while the membrane support 261 is illustrated as being positioned within an interior of the first (proximal) mesh portion 250A, an additional membrane support 261 may be positioned within an interior of the second (distal) mesh portion 250B in some embodiments. In other example embodiments, each mesh portion 250A, 250B may include its own membrane.

### Clauses

Exemplary embodiments are set out in the following numbered clauses:

Clause 1. A vascular plug for treating a patient, comprising: a mesh portion having a radially compressed configuration, a radially expanded configuration, and an interior; a proximal support arm located in a proximal region of the interior of the mesh portion, the proximal support arm including a first proximal hinge; a distal support arm located in a distal region of the interior of the mesh portion, the distal support arm including a first distal hinge; and, a membrane supported by the proximal support arm and the distal support arm within the interior of the mesh portion.

Clause 2. The vascular plug for treating a patient of clause 2, wherein the first distal hinge has a concave upward curve.

Clause 3. The vascular plug for treating a patient of clause 2, wherein the first distal hinge has a concave upward curve.

Clause 4. The vascular plug for treating a patient of clause 1, further comprising a second distal hinge.

Clause 5. The vascular plug for treating a patient of clause 1, further comprising a second proximal hinge.

Clause 6. The vascular plug for treating a patient of clause 1, further comprising a second distal hinge and a second proximal hinge.

Clause 7. The vascular plug for treating a patient of clause 6, wherein the first proximal hinge has a concave downward curve and wherein the second proximal hinge has a concave upward curve.

Clause 8. The vascular plug for treating a patient of clause 7, wherein the first distal hinge has a concave upward curve and wherein the second distal hinge has a concave downward curve.

Clause 9. The vascular plug for treating a patient of clause 8, wherein the second proximal hinge is closer to the membrane than the first proximal hinge, and wherein the second distal hinge is closer to the membrane than the first distal hinge.

Clause 10. The vascular plug for treating a patient of clause 6, wherein the first proximal hinge is at a lower height than the second distal hinge.

Clause 11. The vascular plug for treating a patient of clause 10, wherein the second proximal hinge is at a lower height than the first distal hinge.

Clause 12. The vascular plug for treating a patient of clause 6, wherein the proximal support arm includes a first proximal linkage extending between the first proximal hinge and the second proximal hinge, and a second proximal linkage extending between the second proximal hinge and the membrane.

Clause 13. The vascular plug for treating a patient of clause 12, wherein the distal support arm includes a first distal linkage extending between the first distal hinge and the second distal hinge, and a second distal linkage extending between the second distal hinge and the membrane.

Clause 14. The vascular plug for treating a patient of clause 1, further comprising a central portion for supporting the membrane, wherein the proximal support arm extends from the central portion in a proximal direction, and wherein the distal support arm extends from the central portion in a distal direction.

Clause 15. A vascular plug for treating a patient, comprising: an elongated pusher; a mesh portion connected to a distal end of the elongated pusher, the mesh portion having a radially compressed configuration when constrained in a catheter and a radially expanded configuration when unconstrained; a support frame positioned in an interior of the mesh portion, the support frame having a central portion, a proximal support arm connected to and extending from a proximal side of the central portion, and a distal support arm connected to and extending from a distal side of the central portion; and a membrane fixed to the central portion, the membrane having a radially expanded configuration when the mesh portion is in its radially expanded configuration; wherein the proximal support arm has a first proximal articulating segment and a second proximal articulating segment; and, wherein the distal support arm has a first distal articulating segment and a second distal articulating segment.

Clause 16. The vascular plug for treating a patient of clause 15, wherein the first proximal articulating segment has a concave downward curve, wherein the second proximal articulating segment has a concave upward curve, wherein the first distal articulating segment has a concave upward curve, and wherein the second distal articulating segment has a concave downward curve.

Clause 17. The vascular plug for treating a patient of clause 15, wherein the first proximal articulating segment, the second proximal articulating segment, the first distal articulating segment, and the second distal articulating segment are each comprised of a hinge, a joint, or a spring.

Clause 18. The vascular plug for treating a patient of clause 15, wherein the support frame is formed from only two wires.

Clause 19. A support frame for a vascular plug, comprising: a ring portion for supporting a membrane; a proximal support arm connected to and extending from a proximal side of the ring portion, the proximal support arm having a first dampening means for absorbing force from a flow of blood against the membrane; and a distal support arm connected to and extending from a distal side of the ring portion, the distal support arm having a second dampening means for absorbing force from the flow of blood against the membrane.

Clause 20. The support frame for a vascular plug of clause 19, wherein the first dampening means is comprised of a proximal helical coil, and wherein the second dampening means is comprised of a distal helical coil.

Clause 21. A vascular plug for treating a patient, comprising: a first mesh portion forming a first lobe; a second mesh portion forming a second lobe, and an interconnection area having a reduced diameter compared to the first and second mesh portion.

Clause 22. The vascular plug of clause 21, wherein the first mesh portion has a first braiding density, wherein the second mesh portion has a second braiding density, and wherein the first braiding density is different from the second braiding density.

Clause 23. The vascular plug of clause 22, wherein the first braiding density is greater than the second braiding density.

Clause 24. The vascular plug of clause 22, wherein the first braiding density is lower than the second braiding density.

Clause 25. The vascular plug of clause 21, further comprising a first membrane in an interior of the first mesh portion and a second membrane in an interior of the second mesh portion.

Clause 26. The vascular plug of clause 21, further comprising a membrane in an interior of the first mesh portion.

Clause 27. The vascular plug of clause 21, further comprising a membrane in an interior of the second mesh portion.

Clause 28. The vascular plug of clause 21, wherein the interconnection area comprises a joint.

Clause 29. The vascular plug of clause 21, further comprising a passageway interconnecting the first mesh portion, the second mesh portion, and the interconnection area.

Clause 30. The vascular plug of clause 21, wherein the interconnection area comprises a connector.

Clause 31. The vascular plug of clause 30, wherein the connector comprises a radiopaque marker.

Clause 32. The vascular plug of clause 21, further comprising a support frame in an interior of the first mesh portion and the second mesh portion.

Clause 33. The vascular plug of clause 32, wherein the support frame comprises a membrane support.

Clause 34. The vascular plug of clause 33, wherein the membrane support is positioned within the interior of the first mesh portion.

Clause 35. The vascular plug of clause 32, wherein a distal portion of the support frame comprises a flexible segment.

Clause 36. The vascular plug of clause 35, wherein the flexible segment comprises a coil.

Although the invention has been described in terms of particular embodiments and applications, one of ordinary skill in the art, in light of this teaching, can generate additional embodiments and modifications without departing from the spirit of or exceeding the scope of the claimed invention. Accordingly, it is to be understood that the drawings and descriptions herein are proffered by way of example to facilitate comprehension of the invention and should not be construed to limit the scope thereof.

1-40. (canceled)

41. A vascular occlusion device, comprising:
a mesh portion having a radially compressed configuration and a radially expanded configuration, wherein the mesh portion comprises a first bulb having a first interior and a second bulb having a second interior;
a first membrane positioned within the first interior of the first bulb; and
a second membrane positioned within the second interior of the second bulb.

42. The vascular occlusion device of claim 41, further comprising a support frame positioned within the mesh portion.

43. The vascular occlusion device of claim 42, wherein the support frame includes a first membrane support and wherein the first membrane is supported by the first membrane support.

44. The vascular occlusion device of claim 43, wherein the support frame includes a second membrane support and wherein the second membrane is supported by the second membrane support.

45. The vascular occlusion device of claim 41, wherein the first membrane is substantially the same dimensions as the second membrane.

46. The vascular occlusion device of claim 41, wherein the first membrane is substantially the same shape as the second membrane.

47. The vascular occlusion device of claim 41, further comprising a medial support arm connected between the first membrane and the second membrane.

48. The vascular occlusion device of claim 41, wherein the first membrane and the second membrane are each comprised of a circular shape.

49. A vascular occlusion device, comprising:
a mesh portion having a radially compressed configuration and a radially expanded configuration, wherein the mesh portion comprises a first bulb having a first interior and a second bulb having a second interior;
a support frame located within the mesh portion, the support frame including a first membrane support and a second membrane support;
wherein the first membrane support is positioned within the first interior of the first bulb;
wherein the second membrane support is positioned within the second interior of the second bulb;
a first membrane supported by the first membrane support; and
a second membrane supported by the second membrane support.

50. The vascular occlusion device of claim 49, wherein the support frame comprises a medial support arm connected between the first membrane support and the second membrane support.

51. The vascular occlusion device of claim 50, wherein the medial support arm is comprised of an S-shape.

52. The vascular occlusion device of claim 50, wherein a proximal side of the medial support arm is positioned at a first radial half of the first membrane support, wherein a distal side of the medial support arm is positioned at a second radial half of the second membrane support, and wherein the first radial half is opposite to the second radial half.

53. The vascular occlusion device of claim 50, wherein the support frame comprises a proximal support arm connected to and extending from a proximal side of the first membrane support.

54. The vascular occlusion device of claim 53, wherein the support frame comprises a distal support arm connected to and extending from a distal side of the second membrane support.

55. The vascular occlusion device of claim 49, wherein the first membrane support comprises a first ring and wherein the second membrane support comprises a second ring.

56. The vascular occlusion device of claim 49, wherein the first membrane and the second membrane each comprise a flexible membrane.

57. The vascular occlusion device of claim 49, wherein the first membrane is substantially the same size and shape as the second membrane.

58. A support frame for a vascular plug, comprising:
a first ring portion;
a second ring portion;
a proximal support arm connected to and extending from a proximal side of the first ring portion;
a distal support arm connected to and extending from a distal side of the second ring portion; and
a medial support arm connected between the first ring portion and the second ring portion.

59. The support frame for a vascular plug of claim 58, further comprising a first membrane supported by the first ring portion and a second membrane supported by the second ring portion.

60. The support frame for a vascular plug of claim 58, wherein a proximal side of the medial support arm is positioned at a first radial half of the first ring portion, wherein a distal side of the medial support arm is positioned at a second radial half of the second ring portion, and wherein the first radial half is opposite to the second radial half.
